(19) United States
(12) Patent Application Publication
(10) Pub. No.: US 2019/0066849 A1
(43) Pub. Date: Feb. 28, 2019

(54) Systems and Methods for Improved Patient Care

(71) Applicants: Donald Lawrence, Nashville, TN (US); Blake Burdeen, Nashville, TN (US)

(72) Inventors: Donald Lawrence, Nashville, TN (US); Blake Burdeen, Nashville, TN (US)

(21) Appl. No.: 16/111,035

(22) Filed: Aug. 23, 2018

Related U.S. Application Data
(60) Provisional application No. 62/548,972, filed on Aug. 23, 2017.

(51) Int. Cl. G16H 80/00; G16H 10/60; G16H 20/00; G06N 5/02; G06F 17/27

(52) U.S. Cl. CPC G16H 80/00; G16H 10/60; G16H 20/00; G10L 15/04; G06F 17/2705; G06F 17/274; G06N 5/02

(57) ABSTRACT

Systems and methods for improved patient care are disclosed. A computer-implemented method for improved patient care may include receiving patient data, selecting an interaction mode based on the patient data, and processing the patient data based on an interaction mode. The system and method may include generating a healthcare response based on the processing of the patient data. An apparatus and a non-transitory computer-readable storage medium are also disclosed.

The present application claims the benefit of Provisional Application No. 62/548,972, filed Aug. 23, 2017, the contents of which are incorporated by reference.

### TECHNICAL FIELD

The present invention generally relates to patient healthcare care, and more particularly, to the methods and systems for improving patient care using artificial intelligence.

### BACKGROUND

When a healthcare provider treats a patient, traceable and effective communication between the healthcare provider and the patient may be difficult. For example, a patient and the healthcare provider may have a long and detailed conversation about the patient's health situation. However, the healthcare provider may only record certain notes about the conversation. Even when a healthcare provider uses a conventional computer system to record certain notes during a conversation with the patient, the healthcare provider may be distracted by the very use of the computing platform. These conventional systems and methods are not effective because the healthcare provider's limited notes miss important information that is necessary to treat a patient. From these limited notes, the conventional systems limit both the treatment of the patient and limit the ability to determine whether the treatment of the patient is effective.

Furthermore, conventional systems may have difficulty monitoring and tracking the patient's compliance with prescribed treatment. This failure of conventional systems to effectively monitor and track a patient's compliance includes limitations of the prior art systems that (1) fail to record important notes from the healthcare provider's conversation with a patient, (2) fail to effectively distinguish complex treatment scenarios, and (3) fail to coordinate among multiple healthcare providers who may not communicate with each other during the complex treatment of a patient.

Additionally, when a healthcare provider begins providing care to a patient, the healthcare provider may select and use a clinical pathway that dictates what steps to take when providing the care. A clinical pathway may be a rigid series of steps that the healthcare provider does not deviate from, even if the patient's health situation is somewhat unique. Furthermore, to create a new clinical pathway, a large amount of time, data, and review by other healthcare providers may be needed. The prior art healthcare computer systems and methods fail to develop a unique treatment plan because those systems cannot effectively manage a large amount of data and cannot readily share that data amount various healthcare providers.

### SUMMARY

The present invention generally relates to improving the function and operation of computer-based patient care systems with artificial intelligence (AI) that overcomes the failures of the conventional prior art. More particularly, the present invention relates to a novel application of receiving patient data, determining how a computing system should react to the patient data, combining the patient data with other data, analyzing data to evaluate effectiveness of treatment prescribed, tracking/monitoring patient's compliance with a treatment plan, coordinating patient care among various healthcare providers, managing a large amount of shared data, customizing a treatment plan based upon the patient's healthcare needs, and generating a healthcare response from the data using artificial intelligence. The computing system may react to patient data through a non-conventional selection of an interaction mode or switching from one interaction mode to another. The computing system may select the interaction mode based on a variety of data from non-conventional sources, such as interaction data, context data, scoring data or the like.

The present invention, in one embodiment, contemplates a computer-implemented method for improved patient care. The computer-implemented method may include receiving patient data that is substantially more than a healthcare provider can record during an interaction with a patient. In addition, the present invention may promote automated recordation of the healthcare provider and patient conversation such that the healthcare provider's potential distractions are minimized during the conversations with the patient. The method may include selecting, based on the patient data, an interaction mode. The method may include processing the patient data based on the interaction mode. The method may include generating a healthcare response based on the processing of the patient data.

The present invention, in one embodiment, contemplates an apparatus. The apparatus may include a computer processor and a memory that stores instructions. The instructions, when executed by the processor, may cause the processor to receive patient data. The instructions may cause the processor to select, based on the patient data, an interaction mode. The instructions may cause the processor to process the patient data based on the interaction mode. The instructions may cause the processor to generate a healthcare response based on the processing of the patient data.

The present invention, in one embodiment, contemplates a non-transitory computer-readable storage medium having stored thereon instructions executable by a computer. The computer may execute the instructions to implement a method. The method may include receiving patient data. The method may include selecting, based on the patient data, an interaction mode. The method may include processing the patient data based on the interaction mode. The method may include generating a healthcare response based on the processing of the patient data.

It is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate exemplary embodiments of the invention. Together with the description, they serve to explain the objects, advantages, and principles of the invention. In the drawings:

FIG. 1 is a block diagram of an exemplary system incorporating certain aspects of the present invention.

FIG. 2 is a flowchart illustrating an exemplary method for context data generation.

FIG. 3 is a block diagram of an exemplary context data record.

FIG. 4 is a flowchart illustrating an exemplary method for scoring data generation.

FIG. 5 is a block diagram of an exemplary context scoring model.

FIG. 6 is a block diagram illustrating an exemplary context scoring measurement.

FIG. 7 is a block diagram illustrating an exemplary context scoring result.

### DETAILED DESCRIPTION

Reference will now be made in detail to exemplary embodiments of the invention, some aspects of which are illustrated in the accompanying drawings.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the claims. Furthermore, there is no intention to be bound by any expressed or implied theory presented in the preceding technical field, background, summary, or the following detailed description.

The present invention is directed to systems and methods for improved patient care. In one embodiment, a system or method may facilitate an informational interaction between one or more of a patient, an informal caregiver of the patient (e.g. a family member of the patient), a healthcare provider, and a healthcare computing platform. In one embodiment, a patient may provide interaction data to the healthcare computing platform through a mobile device. The healthcare computing platform may combine the interaction data with other data, such as telemetry data, historical interaction data, medical records, or the like to determine an interaction mode with the patient. Based on the interaction mode, the healthcare platform may generate a healthcare response to send to the patient, put the patient in contact with a healthcare provider, or the like.

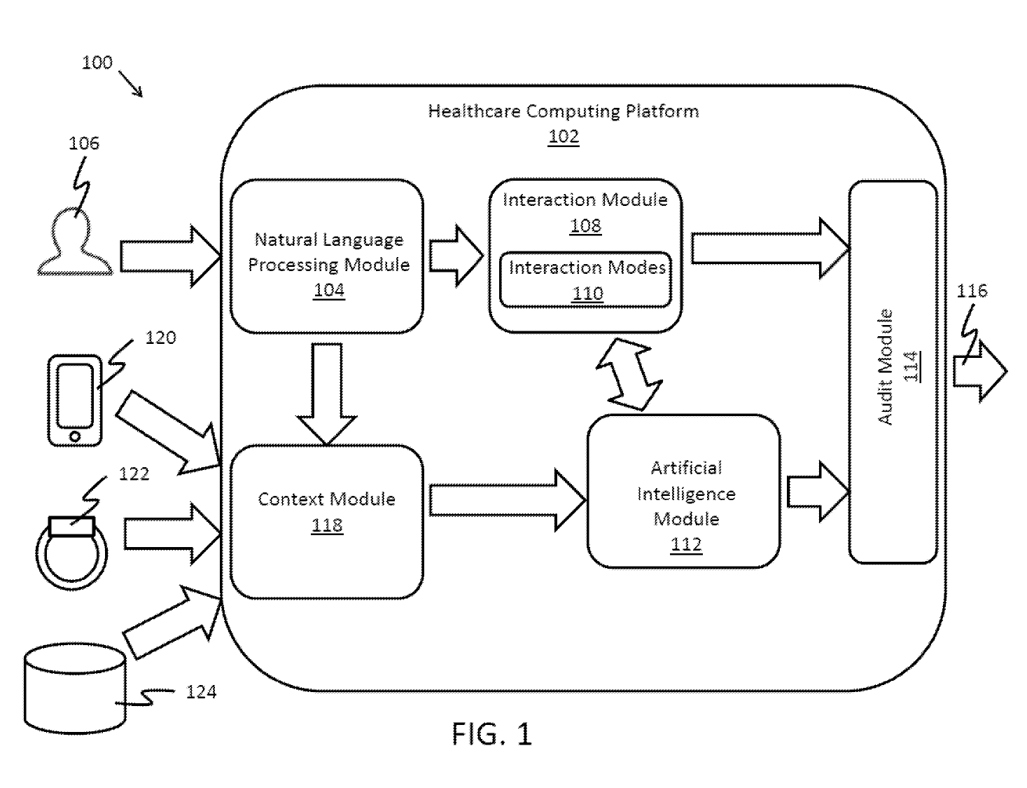

As discussed herein, an action taken by a patient may include an action taken by a caregiver the patient may have, unless specified otherwise. For example, the patient submitting data to the healthcare computing platform may include the patient's caregiver submitting at least a portion of the patient data. FIG. 1 depicts one embodiment of an exemplary system 100 for improved patient care. In one embodiment, the system 100 may include a healthcare computing platform 102. The healthcare computing platform 102 may include one or more computing devices. The one or more computing devices may interact with one another to receive data, process the data, and generate a response. In some embodiments, a computing device may include a computer processor and memory. The memory may store instructions to be executed by the processor. In another embodiment, a computing device may include a non-transitory, computer-readable storage medium. The storage medium may have instructions stored on the medium that are executable by a processor.

In one embodiment, a computing device of the healthcare computing platform 102 may include a server, a database server, a desktop computer, a laptop computer, a tablet, or the like. The one or more computing devices may include one or more modules. The one or more modules may carry out certain functions of the healthcare computing platform 102 as described herein. A module may include software, hardware, or a combination of software and hardware to carry out its one or more functions. In some embodiments, the one or more modules may include a natural language processing (NLP) module 104, an interaction module 108, an artificial intelligence (AI) module 112, an audit module 114, or a context module 118. In some embodiments, the instructions of the memory or computer-readable storage medium may include one or more of the modules.

In one embodiment, the computing healthcare platform 102 may receive patient data. As used herein, “patient data” may include interaction data regarding the patient 106, telemetry data derived from the patient 106, historical interaction data, or the like. As used herein, “interaction data” may include text data, voice data, or other communication data between the patient 106, the healthcare computing platform 102, a healthcare provider, or the like. As used herein, “telemetry data” may include data about the patient 106 collected from or sensed by a variety of devices. In one embodiment, telemetry data may include data from a wearable technology device. A wearable technology device may measure one or more health measurements of a person (e.g. heartrate, body temperature, steps taken, or the like). In one embodiment, telemetry data may include data from a mobile device. The data from the mobile device may include acceleration data, location, positional orientation, or the like. As used herein, “historical interaction data” may include interaction data associated with the patient as part of a previous interaction, conversation, or the like. The patient data may include data derived from the above-mentioned data.

In some embodiments, the patient 106 may input interaction data into a computing device. In some embodiments, the computing device may include a cellular phone, a computer (e.g. a desktop computer, laptop computer, a tablet), or the like. The computing device may send the interaction data to the healthcare computing platform 102. For example, the mobile device may send text data to the healthcare computing platform 102. In some embodiments, the text data may include a short message service (SMS) message, text data from a mobile application, an email, or the like. The mobile device may send voice data to the healthcare computing platform 102. The voice data may include a phone call, voice over IP (VOIP) data, voice data received by an application, or the like. The healthcare computing platform 102 may receive the interaction data from a data network, a cellular network, over the Internet, or the like.

In some embodiments, the healthcare computing platform 102 may include an NLP module 104. The NLP module 104 may receive at least a portion of the patient data from the patient 106. For example, in some embodiments, the NLP module 104 may receive the interaction data from the patient 106. The NLP module 104 may receive the patient data through the healthcare computing platform 102.

In one embodiment, the NLP module 104 may receive the patient data as input, process the patient data, and output processed data. The NLP module 104 may include functions for speech-to-text, voice-recognition, part-of-speech tagging, word segmentation, speech segmentation, terminology extraction, or the like. The processed data may include the interaction data formatted into a format that one or more other components, modules, or the like of the healthcare computing platform 102 can use as input. The NLP module 104 may extract elements from the interaction data into one or more data subsets. The extraction by the NLP module 104 may include multiple levels of granularity ranging from words, phrases, sentences, paragraphs, to the entire interaction session.

In one embodiment, the healthcare computing platform 102 may include an interaction module 108. The interaction module 108 may receive the patient data. The received patient data may include the processed data from the NLP module 104. The interaction module 108 may process the received patient data. The interaction module 108 processing the patient data may include the interaction module 108 selecting, based on processed interaction data, an interaction mode. The interaction module 108 may select from multiple interaction modes 110 based on the received patient data.

In one embodiment, an interaction mode may include one or more procedures that define how patient data 106 is processed by one or more components of the healthcare computing platform 102. An interaction mode may include one or more procedures that define at what point a healthcare provider reviews the patient data, an output from the healthcare computing platform 102, or the like. An interaction mode may include a pre-defined interaction mode or a user-configured interaction mode. A user-configured interaction mode may include an interaction mode configured by a healthcare provider. The healthcare provider may configure the interaction mode based on the patient 106. In some embodiments, the AI module 112 may create or modify an interaction mode based on the processing performed by the AI module 112.

In some embodiments, the interaction module 108 may select an interaction mode from the multiple interaction modes 110 based on one or more factors. In one embodiment, the interaction module 108 may select an interaction mode based on whether the patient 106 is currently being treated for a healthcare problem. In response to the patient 106 not being currently treated, the interaction module 108 may select a first mode. In response to the patient 106 currently being treated, the interaction module 108 may select a second mode. In some embodiments, the interaction module 108 may select a second interaction mode after selecting a first interaction mode based on patient data, data from another module, or the like.

In some embodiments, the interaction module 108 may select an interaction mode based on pre-defined parameters. Pre-defined parameters may include a parameter configured by a healthcare provider of the patient 106, the presence of certain words, phrases, or the like in interaction data from the patient 106, or the like. In one embodiment, selecting the interaction mode may include selecting a direct interaction mode. The direct interaction mode may include a provider-contacting mode. The direct interaction mode may include the healthcare computing platform 102 putting the patient 106 in contact with a healthcare provider through the patient's 106 computing device. For example, the healthcare computing platform 102 may notify the healthcare provider to contact the patient 106. The healthcare computing platform 102 may notify the healthcare provider through an SMS message or a voice message on the healthcare provider's mobile device, a message in a mobile application of the healthcare provider's mobile device, an email, or the like. In one embodiment, the healthcare computing platform 102 may insert the patient 106 into a communication queue of patients waiting to be contacted by a healthcare provider. The healthcare provider may contact the patient 106 through one of a variety of communication methods.

In some embodiments, the interaction module 108 may select the interaction mode based on the content of the interaction data from the patient. In some embodiments, the interaction module 108 may select the interaction mode based on receiving a selection of a mode from the patient 106. For example, the patient 106 may select the direct interaction mode on his or her mobile device, and the mobile device may send the selection to the interaction module 108. In one embodiment, the interaction module 108 may select another interact mode based on interaction data, a selection received from the patient's 106 mobile device, context data, or the like, as explained below.

In some embodiments, the healthcare computing system 102 may include an Artificial Intelligence (AI) module 112. The AI module 112 may include one or more artificial intelligence models, machine learning models, or the like. The AI module 112 may diagnose the patient 106, select relevant healthcare education information, determine the patient's 106 compliance with a clinical pathway, generate modifications to a clinical pathway, determine trends in patient data, determine risk factors, determine contextual meaning for interaction data, or the like.

In one embodiment, the AI module 112 may receive patient data (either directly from the patient 106 or after it has been processed by the NLP module 104, the interaction module 108, or the like), context data, scoring data, or the like as input. The AI module 112 may process the patient data, context data, or scoring data and generate a healthcare response 116 based on that data. In one embodiment, the AI module 112 may receive data from a healthcare provider. The AI module 112 may receive the data from the healthcare provider in the form of context data, scoring data, a clinical pathway, or the like. In one embodiment, the received data from the healthcare provider may include patient profile data, a diagnosis, a prognosis, a treatment plan, healthcare provider-defined content, the healthcare provider's practice, a hospital or care facility, or the like.

In one embodiment, the AI module 112 may process the patient data, context data, scoring data, or the like based on the interaction mode. The interaction module 108 selecting the interaction mode may include selecting a system interaction mode. In one embodiment, the system interaction mode may include the healthcare computing platform 102 interacting with the patient 106 without input from a healthcare provider. In another embodiment, a healthcare response 116 generated in response to the interaction module 108 selecting the system interaction mode may include patient care information, healthcare education information, appointment information, or the like.

In yet another embodiment, the interaction module 108 selecting the interaction mode may include selecting an augmented interaction mode. The augmented interaction mode may include the healthcare computing platform 102 processing data to generate a preliminary healthcare response and receiving input from a healthcare provider regarding the preliminary healthcare response. In one embodiment, the augmented interaction mode may include the context module 118 generating context data and scoring data. The AI module 112 may use the interaction data from the patient 106, the context data, the scoring data, or the like to generate a preliminary healthcare response. The healthcare computing platform 102 may present the preliminary healthcare response, interaction data, context data, scoring data, healthcare database data, or the like to a healthcare provider. The presented data may be organized by one or more health aspects identified in the scoring data. The healthcare provider may consult the preliminary response, interaction data, context data, scoring data, healthcare database data, or the like to approve, reject, or supplement the preliminary response.

In some embodiments, the AI module 112 may generate an estimated accuracy for the preliminary healthcare response. The estimated accuracy may include a confidence of the AI module 112 in the accuracy of the preliminary response. In one embodiment, in response to the estimated accuracy being below a predetermined accuracy threshold, the healthcare computing platform 102 may send the preliminary healthcare response to a healthcare provider. The predetermined accuracy threshold may include a threshold configured by a user. In response to the estimated accuracy being equal to or above the threshold accuracy, the healthcare computing platform 102 may send the preliminary healthcare response as the healthcare response 116 to the patient 106.

In one embodiment, the healthcare computing platform 102 may include an audit module 114. The audit module 114 may store patient data, context data, scoring data, a clinical pathway, an output from a module of the healthcare computing system 102, or the like. Storing the data may include storing the data in one or more databases. In some embodiments, historical interaction data may include interaction data stored by the audit module 114.

In one embodiment, the healthcare computing platform 102 may generate a healthcare response 116. The healthcare computing platform 102 may generate the healthcare response 116 based on the processing of the patient data, context data, scoring data, a clinical pathway, or the like. In some embodiments, the healthcare computing platform 102 may send the healthcare response 116 to the patient 106, a healthcare provider, or the like. The healthcare response 116 may include a variety of responses. The healthcare response 116 may include a diagnosis, healthcare education information, appointment information, a task for the patient 106 to perform, or the like. In one embodiment, the healthcare response 116 may include the healthcare computing platform 102 sending data to the patient 106 periodically. For example, this may include a daily request for the patient 106 to send how the patient 106 is feeling that day to the healthcare computing system 102.

In one embodiment, the healthcare computing platform 102 may include a context module 118. The context module 118 may receive input data from one or more sources. The context module may generate context data, scoring data, or the like. As used herein, “context data” may include output data from a context module based on interaction data, telemetry data, historical interaction data, healthcare database data, or the like. As used herein, “scoring data” may include data regarding one or more health aspect scores generated by the context module based on interaction data, telemetry data, historical interaction data, healthcare database data, or the like.

In one embodiment, the context module 118 may receive the input data in response to an interaction between the patient 106 and the healthcare computing platform 102. For example, in response to receiving interaction data from the from the patient 106, the healthcare computing platform 102 may send a request to one or more input data sources for a current version of the input data. In some embodiments, the context module 118 may receive the input data at pre-defined intervals as configured by a user, the healthcare computing platform 10, or the like.

The one or more sources of input data may include a mobile device 120 of the patient 106, a wearable technology device 122 of the patient 106, a healthcare database 124, or the like. It should be noted that although FIG. 1 depicts the healthcare database 124 as one database and that the healthcare database 124 is referred to herein as a single database, the healthcare database 124 may include one or more databases. The one or more databases may hold different types of data. The one or more databases may be owned, operated, controlled, or the like by different entities. For example, multiple healthcare practices may each include an healthcare records database. Each healthcare records database may provide data to the healthcare computing platform 102 to generate context data, scoring data, or the like.

In some embodiments, the data received by the context module 118 may include non-interaction data from the patient 106. In some embodiments, the context module 118 may receive the data, process the data, and output context data or scoring data. The context module 118 may send the output context data or scoring data to another module of the healthcare computing system 102, such as the AI module 112, to use as input. The context data or scoring data may provide context regarding the interaction data to the one or more other modules. In one embodiment, the context module 118 may format the data to be used by the AI module 112. The context module 118 may send the AI module 112 the context data, scoring data, or the like. The AI module 112 may use the context data, scoring data, or the like as input in combination with the processed patient data. The AI module 112 may generate a preliminary healthcare response or healthcare response 116 from the data.

In some embodiments, the interaction module 108 may select the augmented interaction mode or the direct interaction mode after having already selected a system interaction mode. This may be known as “interaction escalation.” In some embodiments, the interaction module 108 may escalate the current interaction in response to various factors. The factors may include analysis (e.g. by the AI module 112, the context module 118, or the like) of patient data, output from the context module 118, trends of patient data, context data, scoring data, or the like. The analysis may include an analysis of these types of data over time. The factors may include trends of extrapolated patient data, context data, scoring data, or the like. The factors may include historical interaction data, the preliminary healthcare response, the healthcare response 116, treatment or care data, pre-defined scenario parameters based on treatment or diagnosis, or the like.

In one embodiment, the healthcare computing platform 102 may send the context data or the scoring data from the context module 118 to the healthcare provider. The data sent to the healthcare provider may include data that is consolidated, summarized, or the like. For example, the healthcare computing platform 102 may send data derived from telemetric data for use during patient-healthcare provider interactions, for use during the healthcare provider approving, rejecting, adjusting, or supplementing, a preliminary healthcare response or healthcare response 116, or the like.

In one example of the system 100, a patient 106 may have a mobile device 120 running a software application in data communication with the healthcare computing platform 102. The patient 106 may enter the text “I want to speak to my doctor” into the application, and the application may send the text data to the healthcare computing platform 102. The NLP module 104 may receive the text data, parse the text data, and output the text data in a format compatible with the interaction module 108. The interaction module 108 may receive the formatted text data. The interaction module 108 may determine, based on the text data including a demand to speak to a healthcare provider, that the text data most closely complies with the selection rules for selecting a direct interaction mode. In response, the healthcare computing platform 102 may log the text data with the audit module 114, and may generate a healthcare response 116. The healthcare computing platform 102 may process historical interaction data, medical records, or the like to determine the identity of the patient's 106 healthcare provider. In response, the healthcare computing platform 102 may notify the patient's 106 healthcare provider that the patient 106 wants to communicate with the healthcare provider. The healthcare response 116 may include sending a message to the patient's 106 mobile device 120 stating that a healthcare provider will contact the patient 106. The healthcare response 116 may include sending an email to a healthcare provider. The email may include a way to access the patient's contact information, context data, scoring data, or the like.

In another example, a patient 106 may have a mobile device 120 running a software application in data communication with the healthcare computing platform 102. The patient 106 may speak into his or her mobile device 120 saying, “My throat hurts, I'm coughing a lot, and my nose is running.” The application may receive the voice data and may send the voice data to the healthcare computing platform 102. The NLP module 104 may receive the voice data, parse the voice data, and output the voice data in a format compatible with the interaction module 108. The interaction module 108 may receive the formatted voice data. The interaction module 108 may determine, based on the voice data including one or more symptoms, that the voice data most closely complies with the selection rules for selecting a system interaction mode. In response, the interaction module 108 may send the formatted voice data and the interaction mode to the AI module 112.

The AI module 112 may receive the formatted voice data. The AI module 112 may receive context data from the context module 118. The context module 118 may have generated the context data based on data from the patient's mobile device 120, wearable technology device 122, and from a healthcare database 124. The data may include a temperature of the patient that the wearable technology device 122 recently sensed, location data from the mobile device 120, or the like. The AI module 112 may use the voice data, the context data, or the like to determine a diagnosis. For example, the AI module 112 may determine that the symptoms from the voice data and the context data align with a flu diagnosis. The AI module 112 may also determine, based on the location data, that the patient 106 is in a geographic area with a higher rate than normal of flu diagnoses. Based on these determinations, the AI module 112 may generate a diagnosis of the flu and may send the diagnosis, along with care instructions, to the patient's 106 mobile device 120. The audit module 114 may log the healthcare response, the voice data, the context data, and the like.

In another example, a patient 106 may have a mobile device 120 running a software application in data communication with the healthcare computing platform 102. The patient 106 may speak into his or her mobile device 120 saying, as in the previous example, “My throat hurts, I'm coughing a lot, and my nose is running.” The application and healthcare computing platform 102 may take similar actions as those in the previous example. However, the AI module 112 may determine that the symptoms from the voice data and the context data align with a pneumonia diagnosis. The AI module 112 may calculate an accuracy for the pneumonia diagnosis and determine that the accuracy is below a threshold accuracy. In response, the AI module 112 may generate a preliminary healthcare response with the pneumonia diagnosis, the interaction module 110 may select the augmented interaction mode, and the healthcare computing platform 102 may notify a healthcare provider about the preliminary healthcare response. The healthcare provider may view the preliminary healthcare response, consult with the interaction data, the context data, the scoring data, the healthcare database data, or the like and confirm the diagnosis. In response, the healthcare computing platform 102 may send a healthcare response 116 with the pneumonia diagnosis to the patient's 106 mobile device 120. The audit module 114 may log the preliminary healthcare response, the healthcare response 116, the voice data, the context data, the scoring data, or the like.

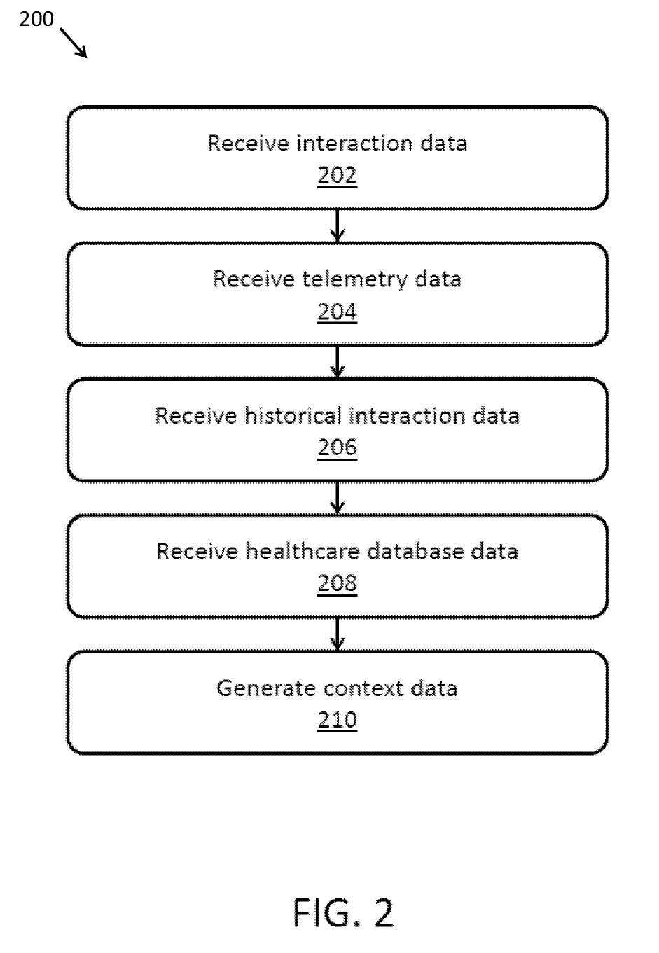

FIG. 2 depicts one embodiment of a method 200 for context data generation. In one embodiment, the method 200 may include receiving 202 interaction data. The context module 118 may receive the interaction data. The interaction data may include the interaction data of the current interaction. The interaction data may include voice data, text data, or the like from the patient's 106 user device as described above. The interaction data may include interaction data that has been processed by the NLP module 104.

In one embodiment, the method 200 may include receiving 204 telemetry data. The context module 118 may receive telemetry data. In some embodiments, the context module 118 may receive telemetry data from the patient's 106 mobile device 120. The telemetry data from the mobile device 120 may include location data, orientation data, acceleration data, or the like. In another embodiment, the context module 118 may receive telemetry data from the patient's 106 wearable technology device 122. A wearable technology device 122 may include a health tracking device. The wearable technology device 122 may measure one or more health measurements of a person (e.g. heartrate, body temperature, steps taken, or the like).

In one embodiment, the method 200 may include receiving 206 historical interaction data. The context module 118 may receive the historical interaction data from the healthcare database 124, the audit module 114, or the like. In one embodiment, the method 200 may include receiving 208 healthcare database data. The context module 118 may receive the healthcare database data from the healthcare database 124. The healthcare database 124 may store healthcare data, interaction data, or other data the healthcare computing platform 102 may use to improve patient care. In some embodiments, the healthcare database 124 may include electronic health records (EHRs), other healthcare data from a healthcare provider, historical healthcare responses, or the like. In one embodiment, the electronic healthcare records may include EHRs for the patient 106 and other patients. In some embodiments, the healthcare database 124 may include a clinical pathway. The clinical pathway may include the clinical pathway chosen by the healthcare provider for treating the patient 106.

In one embodiment, the method 200 may include generating 210 the context data. The context module 118 may generate the context data. The context data may include one or more context data records. The context data may include data extracted from the interaction data, telemetry data, historical interaction data, healthcare database data, or the like and formatted into a format for use by one or more components of the healthcare computing system 102.

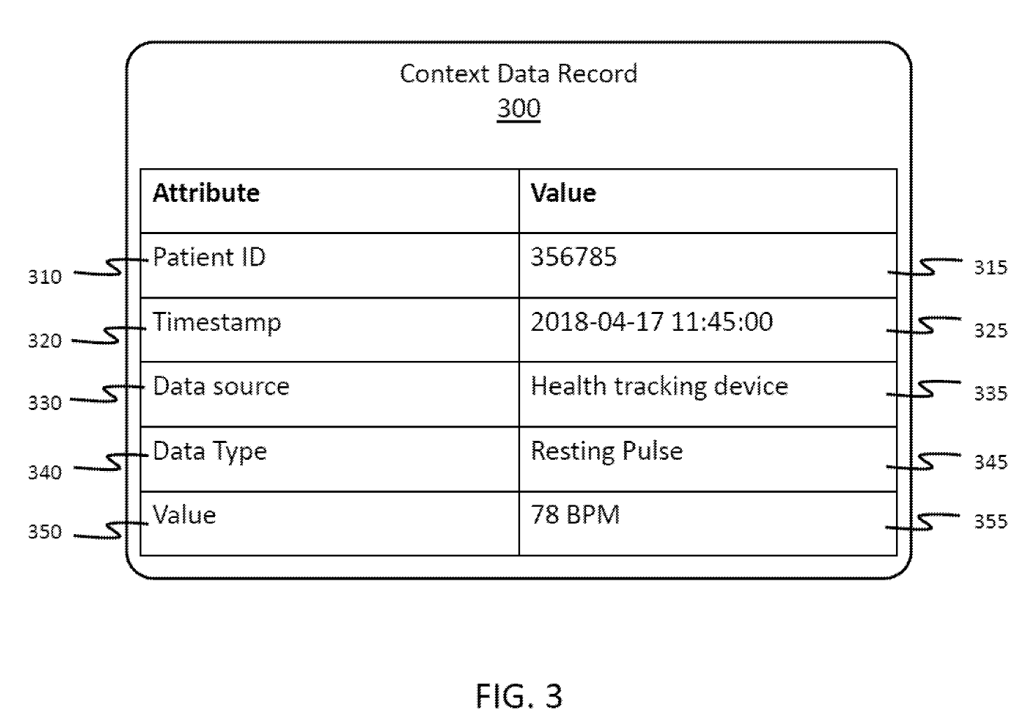

FIG. 3 depicts one embodiment of a context data record 300. In one embodiment, the context data record 300 may include a patient ID attribute 310. The corresponding patient ID value 315 may identify the patient 106. As used herein, an ID value “identifying” a piece of information may include the ID value uniquely identifying the piece of information. The patient ID value 315 may include a number, a string of text, or the like. In one embodiment, the context data record 300 may include a timestamp attribute 320. The corresponding timestamp value 325 may include a date and time the context data record 300 was created, the date and time an event contained in the context data record 300 occurred, or the like. In one embodiment, the context data record 300 may include a data source attribute 330. The corresponding data source value 335 may include the source of the context data for the context data record 300. For example, the data source value 335 may include the patient's 106 mobile device 120, wearable technology device 122, an EHR, or the like. The data source value 335 may include data identifying the data source.

In some embodiments, the context data record 300 may include a measurement attribute 340. The corresponding data type value 345 may include a measurement, interaction data, historical interaction data, an EHR, or the like that the context data record 300 includes. In one embodiment, the context data record 300 may include a value attribute 350. The corresponding value 355 may include a value for the data type included in the context data record. For example, the value 355 may include a heart rate, a temperature, a result of a medical test, a diagnosis, one or more words of interaction data, or the like. In some embodiments, the context data record 300 may include additional data attributes or values.

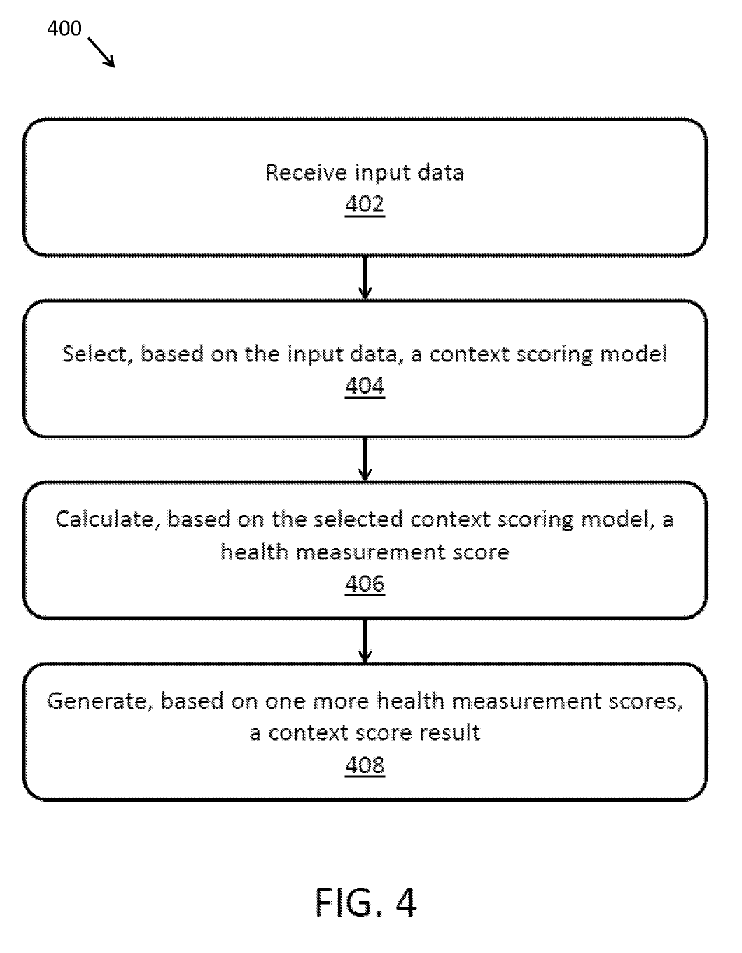

FIG. 4 depicts one embodiment of a method 400 for generating scoring data. The method 400 may include receiving 402 input data. In other embodiments, the input data may include interaction data, telemetry data, historical interaction data, healthcare database data, or the like. Receiving 402 the input data may include similar processes, structures, or the like as steps 202 through 208 of FIG. 2. In one embodiment, the input data may include context data (such as a context data record 300) generated by the context module 118.

In one embodiment, the method 400 may include selecting 404 a context scoring model. In some embodiments, a context scoring model may include one or more data structures, processes, or the like that receive the input data and generate one or more health aspect scores based on the received data. A health aspect score may include a score corresponding to a health aspect category. A health aspect category may include physical, emotional, mental, or the like. A health aspect category may include one or more subcategories. For example, subcategories that may pertain to the physical health aspect category may include cardiovascular, digestive, auditory, or the like. Each subcategory may include one or more measurements. For example, measurements that may pertain to the cardiovascular may include resting pulse, active pulse, blood pressure, or the like. A measurement may pertain to multiple subcategories. A subcategory may pertain to multiple health aspect categories. In one embodiment, a context scoring model may include one or more processes, algorithms, or the like for calculating a score for a health aspect category, subcategory, or measurement.

In some embodiments, selecting 404 a context scoring model may include selecting from a plurality of scoring models. In some embodiments, the context module 118 or the like may select 404 a context scoring model based on the input data. In one embodiment, the context module 118 may select 404 a context scoring model based on the interaction data. The interaction data may include one or more symptoms, bodily organs, or the like, and the context module 118 may select 404 a context scoring model that includes a health aspect category, subcategory, or measurement relevant to that symptom, organ, or the like. In another embodiment, the context module 118 may select 404 a context scoring model based on the context data. The context data may include data requested, used, or the like by the AI module 112.

In some embodiments, the method 400 may include calculating 406 a health measurement score. The health measurement score may include a score based on a measurement of the selected context scoring model. In some embodiments, calculating 406 the health measurement score may include multiplying a base score (based on the patient data, context data, or the like) by a weight for the health aspect category, subcategory, or measurement. The weight for a health aspect category, subcategory, or measurement may be higher in response to the corresponding health aspect category, subcategory, or measurement being more relevant, important, or the like for the score. Calculating 406 the health measurement score may include adding multiple scores from health aspect categories, subcategories, or measurements. In one embodiment, the method 400 may include generating 408 a context scoring result. The context scoring result may include the health aspect category, a subcategory of the health aspect category, a measurement, the context model used to generate the context scoring result, the value of the context scoring result, or the like.

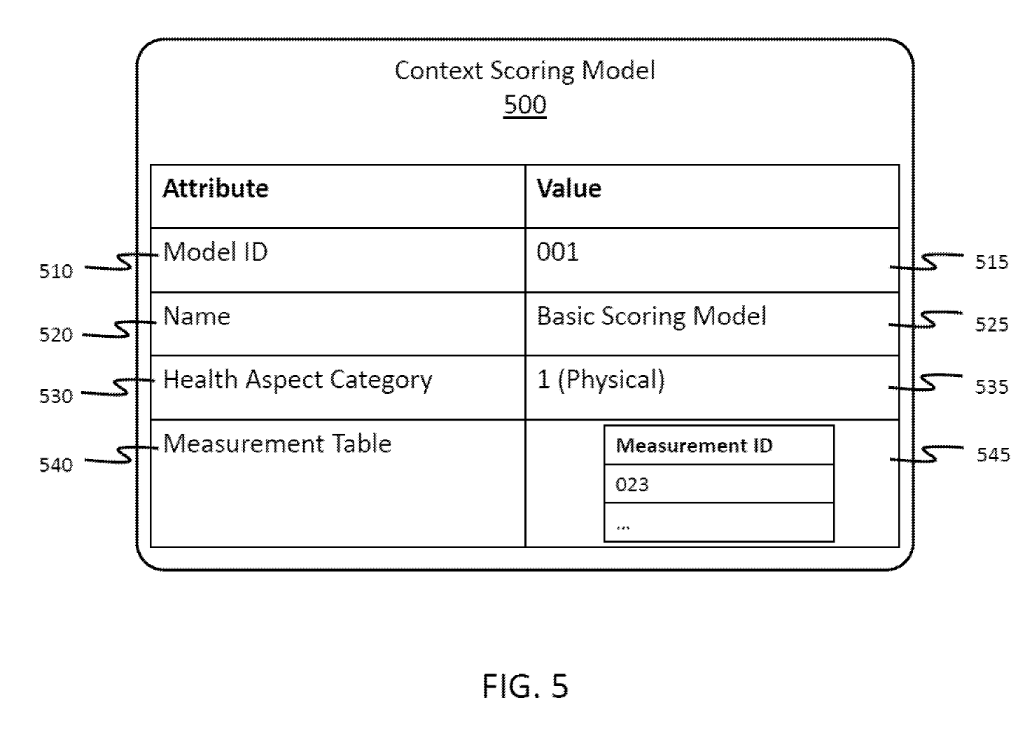

FIG. 5 depicts one embodiment of a context scoring model 500. The context scoring model 500 may include a model ID 510 attribute. The corresponding model ID value 515 may include a value that identifies the context scoring model 500. The context scoring model 500 may include a name attribute 520. The corresponding name value 525 may include a string of text that includes the name of the context scoring model 500. In some embodiments, the context scoring model 500 may include a health aspect category attribute 530. The corresponding health aspect category value 535 may include a value identifying the health aspect category associated with the context scoring model 500. In some embodiments, the context scoring model 500 may include multiple health aspect categories in the health aspect category value 535.

In one embodiment, the context scoring model 500 may include a measurement table attribute 540. The corresponding measurement table value 545 may include a table that may include one or more context scoring measurements. The context scoring measurement may be identified in the measurement matrix value 545 by an ID value. In another embodiment, the context scoring model 500 may include additional data. The additional data may include a version number, a description, an accuracy history, or the like. The accuracy history may include one or more diagnoses and an accuracy for the context scoring model 500 associated with the corresponding diagnosis.

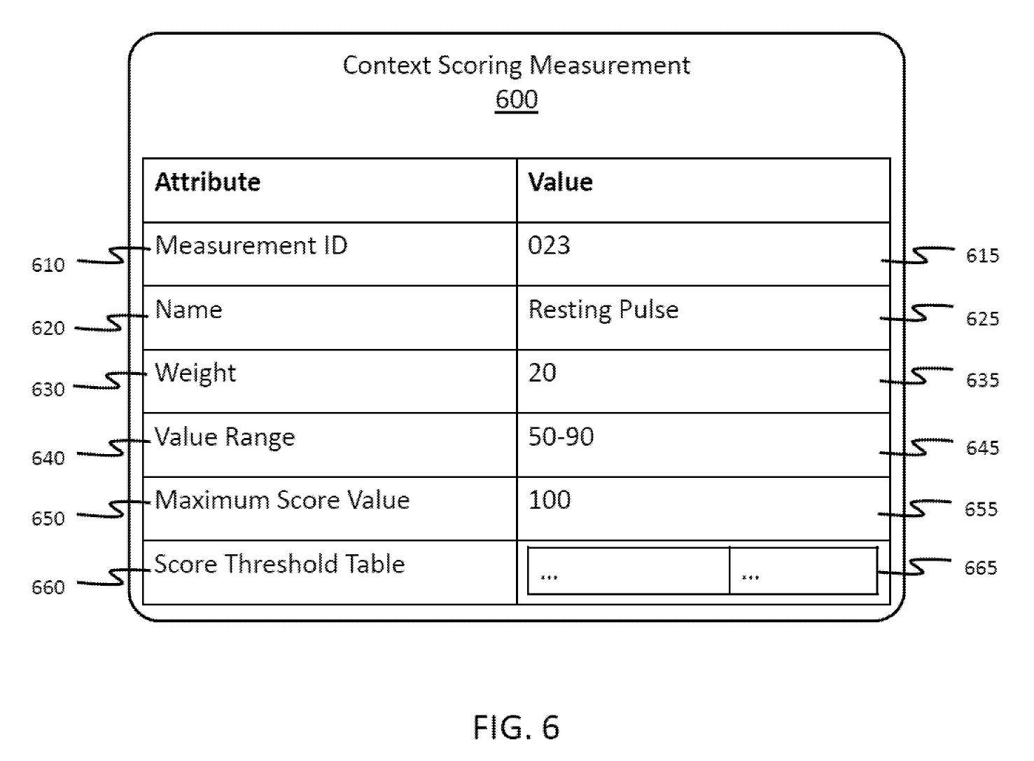

FIG. 6 depicts one embodiment of a context scoring measurement 600. The context scoring measurement 600 may include a measurement that may be included a table entry of the measurement table value 545 of FIG. 5. In one embodiment, the context scoring measurement 600 may include a measurement ID attribute 610. The corresponding measurement ID value 615 may include a value that identifies the context scoring measurement 600. The context scoring measurement 600 may include a name attribute 620, and a corresponding name value 625 may include a string of text that includes the name of the context scoring measurement 600 or the like. In some embodiments, the context scoring measurement 600 may include a weight attribute 630. The corresponding weight value 635 may include a weight value. The weight value 635 may be used to calculate the health aspect score for the measurement type.

In some embodiments, the context scoring measurement 600 may include a value range attribute 640. The corresponding value range value 645 may include a range of values, set of values, or the like the value of the measurement type may be. In one embodiment, the context scoring measurement 600 may include a maximum score value attribute 650. The corresponding maximum score value 655 may include a maximum amount that the scoring result for the context scoring measurement 600 can be.

In some embodiments, the context scoring measurement 600 may include a score threshold table attribute 660. In one embodiment, the corresponding score threshold table value 665 may include a table. An entry in the score threshold table value 665 may include a value of the context scoring measurement and a corresponding score for that value. In some embodiments, the context scoring measurement 600 may include additional data. The additional data may include a score interval value (e.g. a value specifying the precision of the score value), a value specifying whether the score result for the context scoring measurement 600 increases or decreases the context scoring result, or the like.

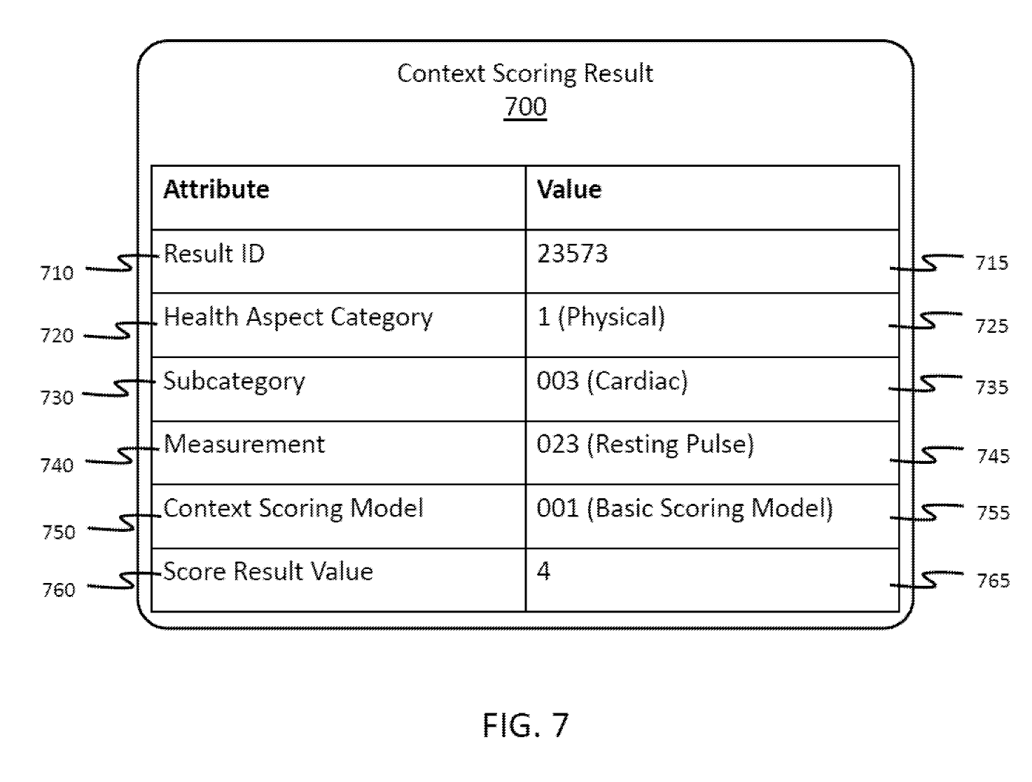

FIG. 7 depicts one embodiment of a context scoring result 700. The context scoring result 700 may include a result ID attribute 710 and a corresponding result ID value 715. The result ID value 715 may identify the context scoring result 700. The context scoring result 700 may include a health aspect category attribute 720 and a corresponding health aspect category value 725. The health aspect category value 725 may include an identifier identifying the health aspect category associated with the context scoring result 700. In some embodiments, the context scoring result 700 may include a subcategory attribute 730, and the corresponding subcategory value 735 may include an identifier identifying the health aspect subcategory associated with the context scoring result 700. The context scoring result 700 may include a measurement attribute 740, and the corresponding measurement value 745 may include one or more measurements used to generate the context scoring result 700. The context scoring result 700 may include a context scoring model 750 attribute, and the corresponding context scoring model value 755 may identify the one or more context scoring models that generated the context scoring result 700. The context scoring result 700 may include a score result value attribute 760, and the corresponding score result value 765 may include the score generated from the one or more scoring context models 755.

In one embodiment, the AI module 112 may receive the one or more context data records 300 or context scoring results 700 from the context module 118. The AI module 112 may process the context data records 300, context scoring results 700, or the like to generate the preliminary healthcare response, the healthcare response 116, or the like. In response, the interaction module 108 may escalate the interaction mode (e.g. from the system interaction mode to the augmented or direct interaction mode).

In some embodiments, the healthcare computing platform 102 may consolidate healthcare data of the patient 106 from multiple sources into a patient data repository. The multiple sources of the patient data may include multiple databases of the healthcare database 124. In one embodiment, the healthcare database 124 may include the patient data repository for the patient 106. In one embodiment, the context module 118 receiving the healthcare database data may include the context module 118 receiving the data from the patient data repository.

In yet another embodiment, the healthcare database 124 may include a clinical pathway. A clinical pathway may include a sequence of one or more medical tasks. The sequence of tasks may branch based on an outcome of a task. The sequence may be ordered based on a chronological order. A task of the clinical pathway may include one or more expected outcomes of medical treatment, one or more medical tests, one or more medications, one or more activities, or the like. As an example, a clinical pathway may indicate that a patient 106 is to take a specified dose of medication and is expected to show improved signs of wellbeing. In response to the patient 106 showing the improved signs of wellbeing, the clinical pathway may indicate to continue taking the medication and the specified dose. In response to the patient 106 not showing improved signs of wellbeing, the clinical pathway may indicate to increase the dosage, prescribe a different medication, or the like.

In one embodiment, the clinical pathway may include a formal clinical pathway. A formal clinical pathway may include a pre-defined clinical pathway, a clinical pathway in use by healthcare providers, or the like. In another embodiment, the clinical pathway may include an informal clinical pathway. An informal clinical pathway may include a dynamically generated clinical pathway, a clinical pathway created, modified, or customized by the AI module 112, or the like.

The clinical pathway may include a clinical pathway selected for the patient 106. In some embodiments, the AI module 112 may select the clinical pathway based on the patient data, context data, scoring data, or the like. In another embodiment, a healthcare provider may select the clinical pathway and may input the selection into the healthcare computing platform 102, the healthcare database 124, or the like.

In one embodiment, the AI module 112 processing the patient data, context data, scoring data, or the like may include the AI module 112 determining a variety of information about the clinical pathway in relation to the patient 106. The AI module 112 may determine a level of compliance of the patient 106 with the clinical pathway. In one embodiment, determining a level of compliance may include the AI module 112 determining whether the patient 106 is following the treatments, taking the medications, performing the activities, or the like of the clinical pathway. In response to the AI module 112 determining that a level of compliance with the clinical pathway is below predetermined threshold, the AI module 112 may modify the clinical pathway. As used herein, “modifying” a clinical pathway may include changing an expected outcome of medical treatment, a medical test, a medical treatment, a medication, an activity, a schedule, a decision tree, or the like. In some embodiments, modifying a clinical pathway may not include selecting a branch of a decision tree that is currently part of the clinical pathway. In one embodiment, modifying a clinical pathway may include modifying the clinical pathway for the specific patient 106 being treated under the clinical pathway.

As an example, the patient 106 may be treated for high blood pressure using a clinical pathway. The healthcare computing platform 102 may periodically request from the patient 106 information about the patient's compliance with the clinical pathway. The healthcare computing platform 102 may ask the patient 106, through the software application of the patient's 106 mobile device, “Have you taken your Spironolactone and stayed in bed today?” The patient 106 may respond with the text data, “Yes.” Location data from the patient's 106 mobile device or health tracking device may indicate that the patient 106 has not stayed in bed during the day. The AI module 112 may receive this interaction data, context data, and the like and may determine that the patient 106 has been in partial compliance with the clinical pathway. In response to the partial compliance, the AI module 112 may determine whether to modify the clinical pathway to require the patient to be admitted to a hospital so that the patient's physical activity can be monitored.

In one embodiment, the AI module 112 may determine whether the actual outcomes of the patient's 106 care match the expected outcomes of the clinical pathway. In response to an actual outcome not matching the expected outcome, the AI module 112 may determine to what extent the actual outcome does not match the expected outcome. The AI module 112 may determine whether to select a certain branch of a decision tree of the clinical pathway, modify the clinical pathway, or the like. For example, a clinical pathway may indicate that a patient's 106 blood pressure should decrease in response to the patient 106 taking Spironolactone. In response to the patient 106 taking the medication but the patient's 106 blood pressure not decreasing, the AI module 112 may determine whether to increase the dosage, use a different medication, or the like.

In some embodiments, in response to the AI module 112 determining to modify the clinical pathway, the AI module 112 may modify the clinical pathway based on the interaction data, context data, scoring data, or the like. In one embodiment, the AI module 112 may modify the clinical pathway based on patient data from multiple patients. The patient data from multiple patients may include one or more EHRs from other patients, one or more outcomes of other patients regarding the same or similar clinical pathways, or the like. The patient data from multiple patients may include aggregated interaction data, context data, scoring data, clinical pathway data, healthcare database data, or the like from multiple patients. The patient data from multiple patients may include diagnoses, treatments, patient demographics, or the like. In some embodiments, the patient data from multiple patients may include data extrapolated from the patient data from the multiple patients. The extrapolated data may indicate one or more trends, patterns, or the like. The AI module 112 may use the above input data to determine how to modify the clinical pathway for the patient 106.

In yet further embodiments, the AI module 112 may generate, based on one or more of the above inputs, an informal clinical pathway component. A clinical pathway component may include an expected outcome of medical treatment, a medical test, a medical treatment, a medication, an activity, a schedule, a decision tree, or the like. The AI module 112 may generate the informal clinical pathway component based on an inference calculation, prediction, or the like of the AI module. The one or more components may provide evidence-based supporting material for the effectiveness and outcome of these modified pathways for clinician, researcher, or peer review. As an example, in one embodiment, the AI module 112 may determine that a patient's 106 treatment for high blood pressuring using Spironolactone is not producing the expected outcomes of the clinical pathway selected for the patient 106. The AI module 112 may determine to modify a clinical pathway for the patient 106. The AI module 112 may use interaction data, context data, scoring data, or the like from the patient 106 as input. The AI module 112 may use patient data from multiple patients as input. The patient data from multiple patients may include medications prescribed, actions taken, therapies attempted in response to Spironolactone not being effective to treat other patients' high blood pressure. The AI module 112 may use this data as input to one or more artificial intelligence models, machine learning models, or the like to generate an informal clinical pathway component. The generated clinical pathway component may include prescribing the patient 106 Nadol.

In one embodiment, a healthcare provider, researcher, the AI module 112, or other component of the healthcare computing platform 102 may measure the effectiveness of an informal clinical pathway generated by the AI module 112. Measuring the effectiveness of the informal clinical pathway may include tracking actual outcomes and comparing them to expected outcomes. In some embodiments, the a healthcare provider, researcher, the AI module 112 may associate one or more informal clinical pathway components with incremental care quality measures that may be based on the various inputs and health aspects tracked by the healthcare computing platform 102 and categorized into standards that can synchronize with existing formal clinical pathways and with the healthcare scoring processes.

In some embodiments, a preliminary healthcare response or a healthcare response 116 may include the modified clinical pathway, the generated informal clinical pathway component, or the like. A healthcare provider may approve, reject, supplement, or modify the modified clinical pathway, generated clinical pathway component, or the like. In one embodiment, the healthcare computing platform 102 may use one or more generated clinical pathway components and formal clinical pathway data to analyze and identify improvements and variations of the decision trees through predictive analysis, data analytics, or the like. The healthcare computing platform 102 may present these finding to a healthcare provider to improve the original clinical pathway, provide a potential new pathway for review, or the like.

As can be seen from the above description, the embodiments of the systems and methods described herein may improve patient care over conventional systems and methods. The systems and methods may generate a healthcare response for treating a patient based not only on information exchanged between the patient and a healthcare providers, but also based on unconventional sources of data such as context data gathered from a variety of devices, scoring data, healthcare database data, or the like. The systems and methods of the present invention may use the gathered data to select an interaction mode for the patient and may use artificial intelligence to analyze the gathered data to generate the healthcare response. The information gathered, the outcomes of treatment, or the like may be recorded for traceability and for later analysis of the effectiveness of the generated healthcare response.

Whereas previous efforts have determined compliance with a clinical pathway based on provider-patient conversations, the present invention may determine compliance based on unconventional sources such as recorded interaction data, the context data, results from artificial intelligence and machine learning systems, or the like. Furthermore, unlike previous efforts, the present invention may generate clinical pathway modifications based on data from the patient, data from multiple patients, trends in the data from multiple patients, or the like. The modifications of the clinical pathway can be analyzed for effectiveness and may provide evidence-based support for formally modifying a clinical pathway in real time, instead of through conventional data-gathering efforts.

The systems and methods described herein may improve the functionality and operation of a computing device. As described herein, the healthcare computing platform, the one or more modules of the healthcare computing platform, or the like may generate a healthcare response, a modification to a clinical pathway, a customized treatment plan or the like based not only on a healthcare provider's input, analysis, suggestions, or the like, but on interaction data from the patient, more complete data from the patient, context data, scoring data, data from multiple sources, and data from multiple patients. The healthcare responses may be more accurate than a healthcare provider's determination because the healthcare response of the healthcare computing platform takes into account data gathered from the variety of sources, coordinates data from multiple healthcare providers, utilizes tracking data to monitor patients' compliance, evaluates patient data to determine effectiveness of treatment, and analyzes information using artificial intelligence, machine learning, and the like.

Although the above discussion has identified some unconventional aspects of the systems and methods disclosed herein, improvements over conventional efforts, and improvements to computing devices and functionality, it should not be understood that these are the only unconventional aspects, improvements or the like.

As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer readable medium(s) having program code embodied thereon.

In some embodiments, a module may be implemented in programmable hardware devices such as field programmable gate arrays, programmable array logic, programmable logic devices or the like. A module may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present disclosure. The computer readable storage medium may include a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network, for example, the Internet, a local area network, a wide area network and/or a wireless network.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages.

Aspects of the present disclosure are described herein with reference to flowchart illustrations or block diagrams of methods, apparatuses, systems, or computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Thus, although there have been described particular embodiments of the present invention of new and useful SYSTEMS AND METHODS FOR IMPROVED PATIENT CARE, it is not intended that such references be construed as limitations upon the scope of this invention. While several embodiments have been described herein, other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A computer-implemented method for improved patient care, the method comprising:
receiving patient data;
selecting, based on the patient data, an interaction mode;
processing the patient data based on the interaction mode; and
generating a healthcare response based on the processing of the patient data.

2. The computer-implemented method of claim 1, wherein receiving the patient data comprises at least one of:
receiving interaction data from the patient;
receiving data from a mobile device of the patient; and
receiving historical interaction data of the patient.

3. The computer-implemented method of claim 1, wherein processing the patient data based on the interaction mode comprises:
receiving, at an artificial intelligence (AI) module, the patient data; and
processing, at the AI module, the patient data.

4. The computer-implemented method of claim 3:
further comprising
receiving, at a natural language processing (NLP) module, at least a portion of the patient data, and
generating, at the NLP module, processed patient data; and
wherein receiving, at the AI module, the patient data comprises receiving, at the AI module, the processed patient data.

5. The computer-implemented method of claim 3:
further comprising generating a health aspect score based on the patient data; and
wherein receiving, at the AI module, the patient data comprises receiving, at the AI module, the health aspect score.

6. The computer-implemented method of claim 3, further comprising:
receiving, at the AI module, data from a healthcare provider; and
processing, at the AI module, the data from the healthcare provider.

7. The computer-implemented method of claim 6, wherein receiving, at the AI module, data from the healthcare provider comprises receiving at least one of:
receiving medical record data of the patient; and
receiving medical record data of a second patient.

8. The computer-implemented method of claim 1, wherein:
selecting, based on the patient data, the interaction mode comprises selecting a healthcare provider-contacting mode; and
processing the patient data based on the interaction mode comprises sending the patient data to a healthcare provider.

9. The computer-implemented method of claim 1, wherein generating the healthcare response comprises notifying a healthcare provider.

10. The computer-implemented method of claim 9, wherein notifying the healthcare provider comprises notifying the healthcare provider in response to an AI module determining that an estimated accuracy for the healthcare response is below a predetermined accuracy threshold.

11. The computer-implemented method of claim 1:
wherein processing, at the AI module, the patient data comprises determining a level of compliance of the patient with a clinical pathway; and
further comprising, in response to determining that the level of compliance is below a predetermined threshold, modifying the clinical pathway.

12. The computer-implemented method of claim 1, further comprising generating a clinical pathway based on:
the patient data;
data received from a plurality of patients; and
data extrapolated from the patient data and the data received from the plurality of patients.

13. An apparatus comprising:
a computer processor; and
a memory storing instructions that, when executed by the processor, cause the processor to
receive patient data;
select, based on the patient data, an interaction mode;
processing the patient data based on the interaction mode; and
generating a healthcare response based on the processing of the patient data.

14. The apparatus of claim 13, wherein the processor processing the patient data based on the interaction mode comprises:
receiving, at an artificial intelligence (AI) module, the patient data; and
processing, at the AI module, the patient data.

15. The apparatus of claim 14:
further comprising
receiving, at a natural language processing (NLP) module, at least a portion of the patient data, and
generating, at the NLP module, processed patient data; and
wherein receiving, at the AI module, the patient data comprises receiving, at the AI module, the processed patient data.

16. The apparatus of claim 14, further comprising:
receiving, at the AI module, data from a healthcare provider; and
processing, at the AI module, the data from the healthcare provider.

17. A non-transitory computer-readable storage medium, having stored thereon instructions executable by a computer, wherein the computer executes the instructions to implement a method comprising:
receiving patient data;
selecting, based on the patient data, an interaction mode;
processing the patient data based on the interaction mode; and
generating a healthcare response based on the processing of the patient data.

18. The computer-readable storage medium of claim 17, wherein processing the patient data based on the interaction mode comprises:
receiving, at an artificial intelligence (AI) module, the patient data; and
processing, at the AI module, the patient data.

19. The computer-readable storage medium of claim 18:
further comprising
receiving, at a natural language processing (NLP) module, at least a portion of the patient data, and
generating, at the NLP module, processed patient data; and
wherein receiving, at the AI module, the patient data comprises receiving, at the AI module, the processed patient data.

20. The computer-readable storage medium of claim 18, further comprising:
receiving, at the AI module, data from a healthcare provider; and
processing, at the AI module, the data from the healthcare provider.
